(19) United States
(12) Patent Application Publication
(10) Pub. No.: US 2021/0282996 A1
(43) Pub. Date: Sep. 16, 2021

(54) ARM POSITIONING CRADLE FOR SUPINE AND BEACH CHAIR POSITION

(71) Applicant: BONE FOAM, INC., Corcoran, MN (US)

(72) Inventors: Benjamin J. Watters, III, Saint Louis Park, MN (US); Clinton J. McCullough, Blaine, MN (US); Peter A. Cole, JR., North Oaks, MN (US)

(21) Appl. No.: 17/182,940

(22) Filed: Feb. 23, 2021

Related U.S. Application Data
(60) Provisional application No. 62/988,147, filed on Mar. 11, 2020.

(51) Int. Cl. A61G 13/12

(52) U.S. Cl. CPC A61G 13/1235; A61F 5/373; A61G 2200/34; A61G 2200/327

(57) ABSTRACT

A patient positioning system for positioning a patient in preparation for an anterior cervical spinal procedure includes a base section, an upper body support attachable to a superior portion of the base section and a lower body support attachable to an inferior portion of the base section. The upper body support is configured to support the head and upper torso of the patient in a manner that aids in opening cervical spine disk space. The lower body support is configured to comfortably lift and support the legs of the patient. A traction strap assembly is attachable to the base section and configured to extend from the base section up around the shoulders of the patient and along the anterior side of the patient to a lower terminal end to enable intermittent traction of the patient's shoulders.

### CROSS-REFERENCE TO RELATED APPLICATION

This application claims the benefit of U.S. Provisional Application No. 62/988,147, filed Mar. 11, 2020, which is incorporated by reference in its entirety.

### BACKGROUND

Proper positioning of patients during and in preparation for surgery is extremely important in order to provide good operating conditions and effective access to the operative site. During surgery, patients may be placed in positions that are not completely physiologically stable or comfortable, and need to be stabilized and maintained in those positions for considerable amounts of time. Improper positioning of the patient can lead to injury and potential disability and functional loss.

Several surgical procedures place the patient in the supine or beach chair position. During certain of these procedures, one or both arms may need to be supported and positioned so as not to fall within the operative field. The arm or arms are often taped and padded with an assortment of pillows, towels, and/or other simple materials. This preparation takes up valuable time in the Operating Room and is not always successful. The inconsistency and variability of the ad-hoc setup can put the patient at risk for soft tissue injuries and/or neuropathy.

Accordingly, there is an ongoing need for effective patient positioning devices. In particular, there is an ongoing need for support devices capable of effectively positioning a patient's arm during surgery in a consistent and safe manner and that also allow for easy and relatively rapid setup in the Operating Room.

### SUMMARY

Described herein are patient positioning devices configured for supporting and positioning a patient's arm in preparation for and/or during a surgical procedure. Arm support devices described herein can beneficially provide effective support and positioning of a patient's arm in a safe manner and with efficient setup and use. Arm support devices described herein may be utilized to support the arm(s) in a position outside of the operative field in surgical procedures where the patient is in the supine or beach chair position, for example.

In one embodiment, an arm support device includes a bottom section and a separate top section. The bottom section is configured for placement upon the patient's torso. The patient's arm may then be positioned across the bottom section. The top section may then be placed over the arm so that the arm is positioned between the bottom section and the top section.

The bottom section includes a first upper arm portion that extends from a first elbow portion downward to a proximal end, a first lower arm portion that connects to the first elbow portion and extends downward therefrom to a distal end, and a first channel that extends along an inner surface of the bottom section. The first channel is configured in size and shape for receiving and supporting an arm of the patient. The top section includes a second upper arm portion that extends from a second elbow portion downward to a proximal end, a second lower arm portion that connects to the second elbow portion and extends downward therefrom to a distal end, and a second channel that extends along an inner surface of the top section. The second channel is configured in size and shape to sit upon the arm of the patient opposite the bottom section such that the arm of the patient is disposed therebetween.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, in which:

FIGS. 1A and 1B illustrate examples of a conventional approach to positioning an arm across the patient's torso during a surgical procedure;

FIG. 2A is a left side view illustrating an exemplary arm support device in an orientation that permits a patient's left arm to be supported and positioned thereby;

FIG. 2B is a right side view illustrating the exemplary arm support device in an opposite orientation that permits a patient's right arm to be supported and positioned thereby;

FIG. 3 is an exploded side view illustrating an exemplary arm support device according to the invention;

FIG. 4 is an exploded rear elevation view of the exemplary arm support device;

FIG. 5 is an exploded front elevation view of the exemplary arm support device;

FIG. 6 is a top plan view of the exemplary arm support device;

FIG. 7 is a bottom plan view of the exemplary arm support device;

FIG. 8 is a right side elevation view of the exemplary arm support device; and

FIG. 9 is a left side elevation view of the exemplary arm support device.

### DETAILED DESCRIPTION

### Introduction

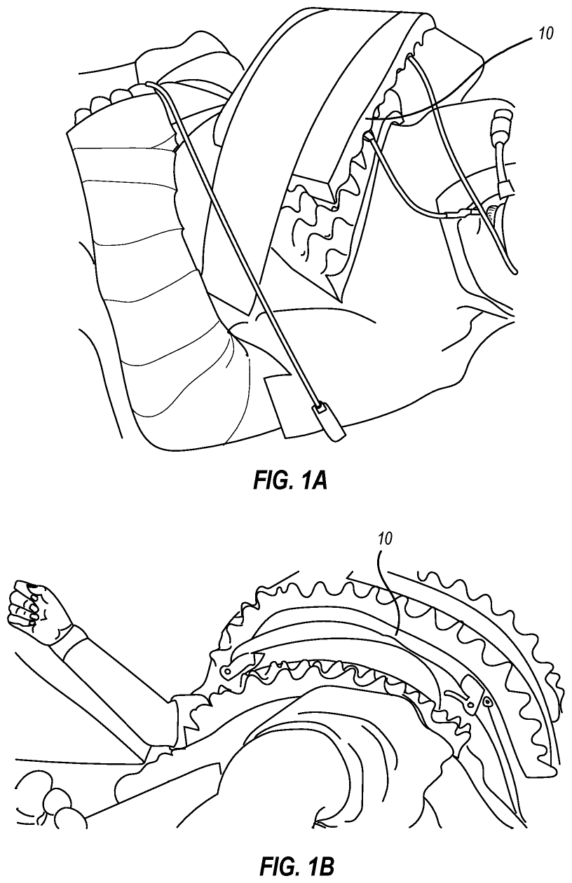

FIGS. 1A and 1B illustrate examples of a conventional approach to positioning an arm across the patient's torso during surgery, showing positioning of the right arm and left arm, respectively. As shown, a patient's left arm 10 is bent across the midline (via horizontal adduction of the shoulder) and secured in position on the patient's torso. This is done to keep the arm out of the operative field. The surgical procedure may be related to the patient's shoulder region, other arm, side, neck, or other region of the body where it is beneficial to keep the arm 10 secured and out of the way, for example.

As shown, the arm 10 is often secured in position using simple padded cutouts, surgical tape, and perhaps other off-the-shelf materials (e.g., towels, pillows, rolled up bedsheets) in an ad-hoc manner. This inconsistent approach is not standardized and can lead to improper positioning of the arm 10. Improper positioning can be associated with soft tissue damage and/or neuropathy, particularly because the position may be held for relatively long periods of time while the patient is unconscious and unable to register impingements or other pain warnings.

Moreover, the non-standardized and ad-hoc approach uses valuable time in preparing the patient for the surgical procedure. Every minute within the Operating Room increases personnel and resource costs. Thus, a standardized approach to arm positioning and a readily assembled device could provide significant cost saving benefits in addition to the improvements to patient safety.

### Arm Support Device Overview

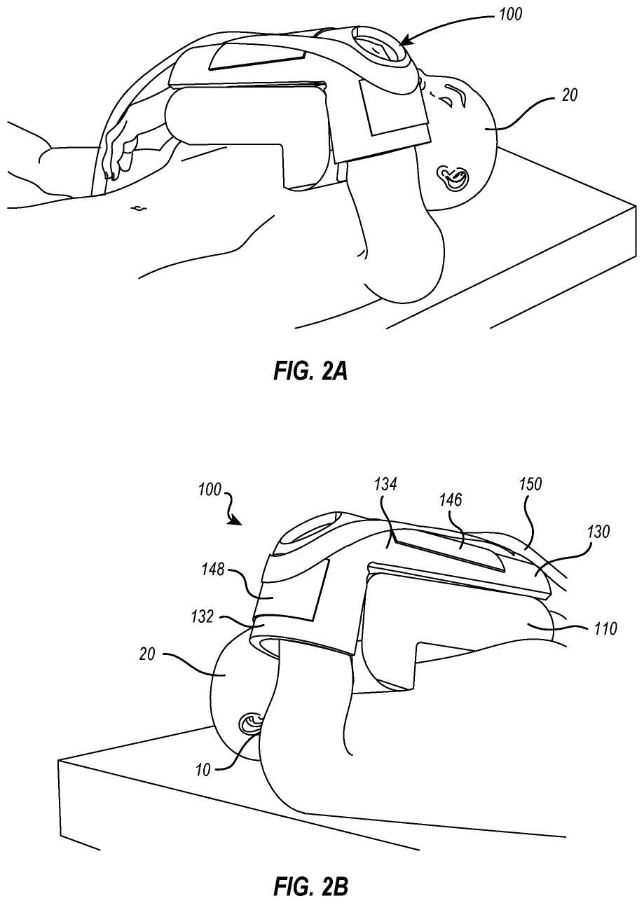

FIGS. 2A and 2B illustrate an exemplary arm support device 100 in use by a patient 20, with FIG. 2A showing the device in use to support the left arm and FIG. 2B showing the device in use to support the right arm. Although the patient 20 is shown here in the supine position, the support device 100 may be suitably used in other patient positions as well, such as the “beach chair position”. Arm support devices described herein can beneficially provide effective support and positioning of a patient's arm in a safe manner and with efficient setup and use.

Referring to the right side view of FIG. 2B, the support device 100 more particularly includes a bottom section 110 and a top section 130. The bottom section 110 can be placed over the patient's torso. The patient's arm 10 may be positioned on top of the bottom section 110 and the top section 130 placed over the arm. In this way, the bottom section 110 lifts and supports the patient's arm 10 while the top section 130 fits over the arm 10 to hold and protect the arm 10 between the bottom section 110 and the top section 130.

The support device 100 may also include a strap assembly 150 that is attachable to the top section 130. The strap assembly 150 is designed to permit operating room staff to apply arm traction by pulling on the strap assembly 150. For example, a proximal strap connector 148 may be disposed on an outer surface of an upper arm portion 132 of the top section 130. A strap can thus engage with the proximal strap connector 148 such that pulling the strap in the distal direction (i.e., the direction extending along the arm toward the hand and beyond) pulls the upper arm portion 132 distally against the patient's upper arm to secure the upper arm and prevent it from falling posteriorly (i.e., prevent horizontal abduction of the shoulder).

The illustrated top section 130 also includes a distal strap connector 146 disposed on an outer surface of a lower arm portion 134. A strap can thus engage with the distal strap connector 146 such that pulling the strap in a posterior (e.g., downward) direction pulls the lower arm portion 134 down against the patient's lower arm to assist in securing the lower arm against the bottom section 110.

As shown, the same strap assembly 150 (e.g., same strap) may be connected to both the proximal strap connector 148 and the distal strap connector 146. Pulling of the strap in a direction with both distal and posterior components can thereby pull the patient's upper arm distally and the patient's lower arm posteriorly against the bottom section 110 simultaneously. In alternative embodiments, multiple straps (not shown) may be utilized. The strap assembly 150, proximal strap connector 148, and/or distal strap connector 146 may comprise hook and loop fastener components, clasps, buckles, or other fastening hardware as known in the art.

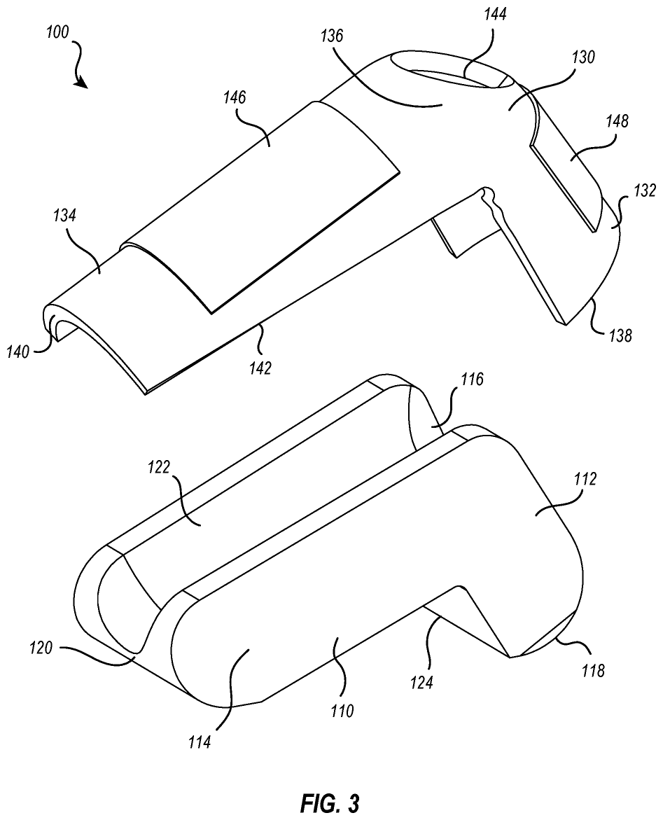

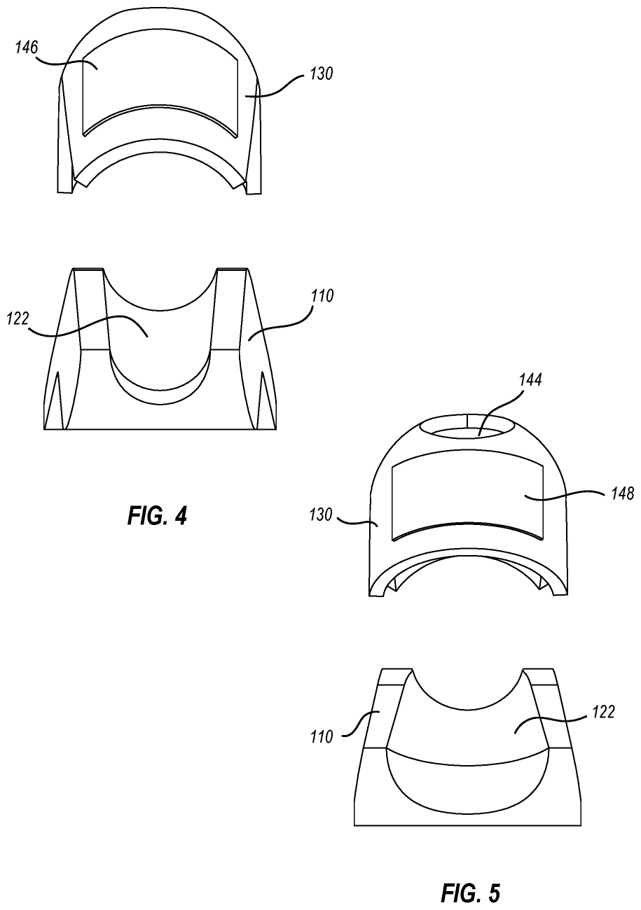

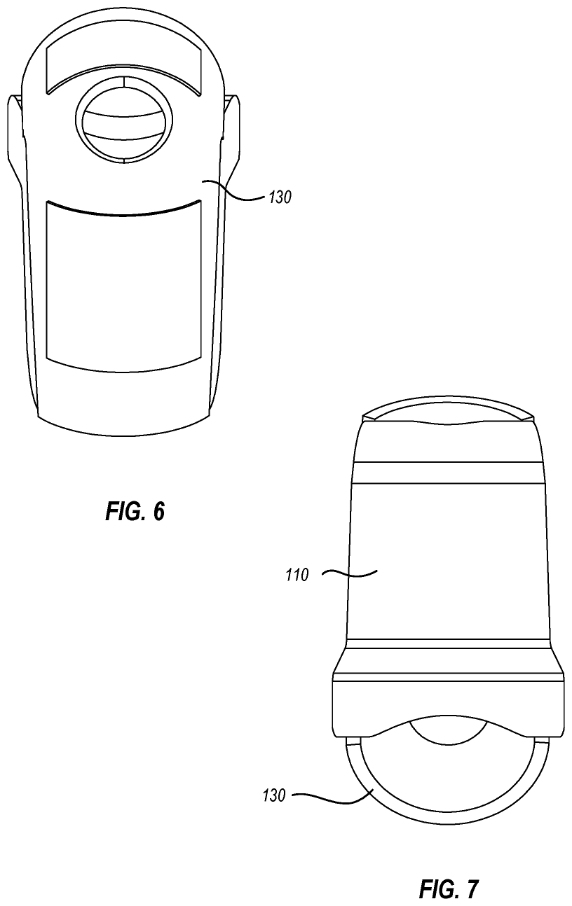

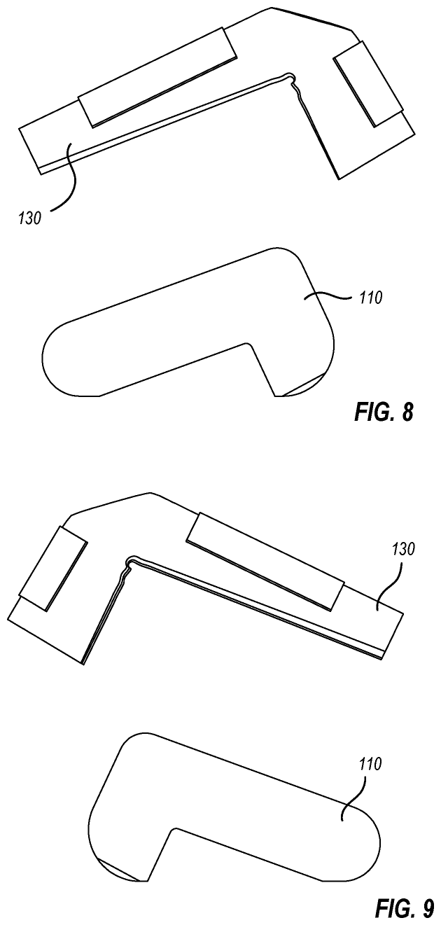

FIGS. 3-9 illustrate additional views of the exemplary arm support device 100. FIG. 3 is an exploded side view; FIG. 4 is an exploded rear elevation view; FIG. 5 is an exploded front elevation view; FIG. 6 is a top plan view; FIG. 7 is a bottom plan view; FIG. 8 is a right side elevation view; and FIG. 9 is a left side elevation view. FIGS. 3-9 show the bottom section 110 and top section 130 spaced apart somewhat to better represent some of the internal features of the device 100.

The bottom section 110 includes an upper arm portion 112 (i.e., for supporting the patient's upper arm) and a lower arm portion 114 (i.e., for supporting the patient's lower arm). These are sometimes referred to herein as “first” upper arm portion 112 and “first” lower arm portion 114 to distinguish them from “second” upper arm portion 132 and “second” lower arm portion 134 of the top section 130). The upper arm portion 112 and lower arm portion 114 are joined together at a “first” elbow portion 116. As shown, the upper arm portion 112 extends downward from the elbow portion 116 to a proximal end 118, while the lower arm portion 114 extends downward from the elbow portion 116 to a distal end 120.

The bottom section 110 includes a “first” channel 122 that extends along an inner surface of the bottom section 110 and is configured in size and shape to receive and support the patient's arm. The channel 122 is shown here as having a rounded, curved profile that extends along the length of both the upper arm portion 112 and lower arm portion 114. Other embodiments may have a different shape for the channel 122 and/or in some embodiments the channel 112 may only extend along a portion of the inner surface of the bottom section 110, such as only across the upper arm portion 112 or only across the lower arm portion 114.

With respect to the top section 130, the upper arm portion 132 joins to the lower arm portion 134 at a “second” elbow portion 136. The upper arm portion 132 extends downward from the elbow portion 136 to a proximal end 138, while the lower arm portion 134 extends downward from the elbow portion 136 to a distal end 140.

The top section 130 includes a “second” channel 142 that extends along an inner surface of the top section 130 and is configured in size and shape to sit upon the arm of the patient opposite the bottom section such that the arm of the patient is disposed therebetween. The channel 142 is shown here as having a rounded, curved profile that extends along the length of both the upper arm portion 132 and lower arm portion 134. Other embodiments may have a different shape for the channel 142 and/or in some embodiments the channel 142 may only extend along a portion of the inner surface of the top section 130, such as only across the upper arm portion 132 or only across the lower arm portion 134.

The outer surface of the top section 130 preferably has a low, rounded profile to reduce the overall size of the device. In contrast, the bottom section 110 has a bottom surface that extends downward from the inner surface to give the bottom section 110 sufficient height to lift and support the patient's arm. That is, the bottom section 110 provides space between the patient's torso and the patient's arm so that the arm can be comfortably and safely positioned. The bottom surface also includes a cutout 124, rather than a flat profile, so that the proximal end 118 and the distal end 120 act as the major contact surfaces against the patient's torso. This beneficially allows for more stable positioning of the bottom section 110 on the patient's torso. In contrast, for example, a substantially flat bottom surface would tend to rock upon a rounded torso shape.

The lower arm portion 134 is somewhat longer than the lower arm portion 114. Because the bottom section 110 abuts the inside of the elbow while the top section 130 abuts the outside of the elbow, the greater length of the lower arm portion 134 relative to the lower arm portion 114 allows the lower arm portion 134 to extend farther proximally to wrap around the outer elbow and outer part of the patient's upper arm. This provides effective coverage of the patient's elbow while still allowing the lower arm portion 134 to be substantially coincident with the corresponding lower arm portion 114. Put differently, the distal ends 120 and 140 are preferably substantially aligned when the device is positioned on the patient so that the patient's lower arm can be supported on both upper and lower sides along a sufficient length.

In the illustrated bottom section 110, the lower arm portion 114 has a greater length than the upper arm portion 112. For example, the lower arm portion 114 may have a length that is about 1.2 to about 2 times the length of the upper arm portion 112, or about 1.4 to about 1.8 times the length of the upper arm portion 112. The length differential beneficially allows the patient's arm to be positioned in a comfortable, safe position with the patient's elbow appropriately distanced from the torso and with good support and angling of the patient's lower arm therefrom.

The upper arm portion 112 and lower arm portion 114 are joined together at an angle of about 90 degrees. Other embodiments may join the upper arm portion 112 and lower arm portion 114 at a somewhat smaller or larger angle. For example, the upper arm portion 112 and lower arm portion 114 may be joined together at an angle of about 60 to about 120 degrees, or about 75 to about 105 degrees. These attachment angles beneficially function to support the arm at a comfortable position while providing enough elbow flexion to keep the arm out of the operative field.

The top section 130 has some features similar to the bottom section 110 so that the patient's arm can be effectively positioned between the two components. The lower arm portion 134 has a length greater than a length of the upper arm portion 134. For example, the lower arm portion 134 may have a length that is about 1.5 to about 2.5 times the length of the upper arm portion 132, or about 1.75 to about 2.25 times the length of the upper arm portion 132.

The upper arm portion 132 and lower arm portion 134 may join together at an angle that is substantially similar to the angle at which the like components of the bottom section 110 join together, such as an angle of about 60 to about 120 degrees, or about 75 to about 105 degrees, or about 90 degrees.

As shown, the top section 130 also includes a cutout 144 configured to receive and surround the patient's elbow. The cutout 144 beneficially allows for some movement of the elbow joint and avoids uncomfortable abrasive rubbing against the patient's elbow.

As best shown in FIG. 4, the bottom section 110 is wider at the proximal end 118 than at the distal end 120, and may progressively taper in width from the proximal end 118 to the distal end 120. Similarly, the top section 130 is wider at the proximal end 138 than at the distal end 140, and may progressively taper in width from the proximal end 138 to the distal end 140. The channels 122 and/or 142 may have a corresponding tapering width. Such tapering aligns with arm anatomy and beneficially allows for better cradling and support of the arm.

The bottom section 110 and top section 130 may be formed from the same or from different materials. Preferably, the bottom section 110 and/or top section 130 are formed from a polymer foam material, such as an open or closed cell foam, a “memory foam” (e.g., low-resilience polyurethane foam and/or other viscoelastic foams), rubber materials, gel padding, or combinations thereof. Some embodiments may also include a water-resistant coating, such as a flexible, water-resistant polymer (e.g., vinyl-based) that coats the foam material and allows for easy cleaning of the device.

### Conclusion

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, or less than 1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an,” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. An arm support device, comprising:
a bottom section that includes
a first upper arm portion that extends from a first elbow portion downward to a proximal end,
a first lower arm portion that connects to the first elbow portion and extends downward therefrom to a distal end, and
a first channel that extends along an inner surface of the bottom section, the first channel being configured in size and shape for receiving and supporting an arm of the patient; and
a top section that includes
a second upper arm portion that extends from a second elbow portion downward to a proximal end,
a second lower arm portion that connects to the second elbow portion and extends downward therefrom to a distal end, and
a second channel that extends along an inner surface of the top section, the second channel being configured in size and shape to sit upon the arm of the patient opposite the bottom section such that the arm of the patient is disposed therebetween.

2. The device of claim 1, wherein the bottom section includes a bottom surface with a cutout between the distal end and the proximal end.

3. The device of claim 1, wherein the bottom section is wider at the proximal end than at the distal end.

4. The device of claim 3, wherein the bottom section progressively tapers in width from the proximal end to the distal end.

5. The device of claim 1, wherein the first lower arm portion has a greater length than the first upper arm portion.

6. The device of claim 5, wherein the first lower arm portion has a length that is about 1.2 to about 2 times the length of the first upper arm portion, or about 1.4 to about 1.8 times the length of the first upper arm portion.

7. The device of claim 1, wherein the first upper arm portion and the first lower arm portion join together at an angle of about 60 to about 120 degrees.

8. The device of claim 7, wherein the first upper arm portion and the first lower arm portion join together at an angle of about 75 to about 105 degrees.

9. The device of claim 1, wherein the top section is wider at the proximal end than at the distal end.

10. The device of claim 1, wherein the top section progressively tapers in width from the proximal end to the distal end.

11. The device of claim 1, wherein the second lower arm portion has a greater length than the second upper arm portion.

12. The device of claim 11, wherein the second lower arm portion has a length that is about 1.5 to about 2.5 times the length of the second upper arm portion, or about 1.75 to about 2.25 times the length of the second upper arm portion.

13. The device of claim 1, wherein the second upper arm portion and the second lower arm portion join together at an angle of about 60 to about 120 degrees.

14. The device of claim 13, wherein the second upper arm portion and the second lower arm portion join together at an angle of about 75 to about 105 degrees.

15. The device of claim 1, wherein the second elbow portion includes a cutout configured for receiving an elbow of the patient.

16. The device of claim 1, wherein the top section further comprises a proximal strap connector disposed on an outer surface of the second upper arm portion.

17. The device of claim 16, wherein the top section further comprises a distal strap connector disposed on an outer surface of the second lower arm portion.

18. The device of claim 17, further comprising a strap assembly configured to attach to one or more strap connectors and to extend therefrom to enable the application of traction to the arm support device.

19. An arm support device, comprising:
a bottom section that includes:
a first upper arm portion that extends from a first elbow portion downward to a proximal end,
a first lower arm portion that connects to the first elbow portion and extends downward therefrom to a distal end, and
a first channel that extends along an inner surface of the bottom section, the first channel being configured in size and shape for receiving and supporting an arm of the patient;
a top section that includes:
a second upper arm portion that extends from a second elbow portion downward to a proximal end,
a second lower arm portion that connects to the second elbow portion and extends downward therefrom to a distal end,
a second channel that extends along an inner surface of the top section, the second channel being configured in size and shape to sit upon the arm of the patient opposite the bottom section such that the arm of the patient is disposed therebetween, and
at least one of a proximal strap connector disposed on an outer surface of the second upper arm portion or a distal strap connector disposed on an outer surface of the second lower arm portion.

20. An arm support device, comprising:
a bottom section that includes:
a first upper arm portion that extends from a first elbow portion downward to a proximal end,
a first lower arm portion that connects to the first elbow portion and extends downward therefrom to a distal end, and
a first channel that extends along an inner surface of the bottom section, the first channel being configured in size and shape for receiving and supporting an arm of the patient;
a top section that includes:
a second upper arm portion that extends from a second elbow portion downward to a proximal end,
a second lower arm portion that connects to the second elbow portion and extends downward therefrom to a distal end,
a second channel that extends along an inner surface of the top section, the second channel being configured in size and shape to sit upon the arm of the patient opposite the bottom section such that the arm of the patient is disposed therebetween,
at least one of a proximal strap connector disposed on an outer surface of the second upper arm portion or a distal strap connector disposed on an outer surface of the second lower arm portion, and
a strap assembly configured to attach to at least one of the proximal or distal strap connectors and to extend therefrom to enable the application of traction to the arm support device.
